(19) United States
(12) Patent Application Publication
(10) Pub. No.: US 2025/0319262 A1
(43) Pub. Date: Oct. 16, 2025

(54) Nebulizer and Its nebulization Module

(71) Applicant: Dongguan Aidisy Machinery & Electronic Equipment Co., Ltd, Dongguan (CN)

(72) Inventor: Jianjun Wang, Dongguan (CN)

(21) Appl. No.: 19/077,095

(22) Filed: Mar. 12, 2025

Related U.S. Application Data
(63) Continuation of application No. PCT/CN2024/087729, filed on Apr. 15, 2024.

(51) Int. Cl. A61M 11/00

(52) U.S. Cl. CPC A61M 11/005; A61M 2205/0211; A61M 2205/0233; A61M 2205/0294

(57) ABSTRACT

This utility model patent discloses a nebulizer and its nebulization module, including a base, a flexible sleeve and a piezoceramic transducer. The flexible sleeve is attached on the edge of the piezoceramic transducer. The base has a mounting chamber where the flexible sleeve is installed. On the outer wall of the base there is an electrode holder. The conductive pins in the holder are connected to the piezoceramic transducer according to the polarity. The edge of the piezoceramic transducer is enclosed by the flexible sleeve to form an integrated structure. The flexibility of the flexible sleeve allows itself to be mounted in the base, achieving a stable connection between the piezoceramic transducer and the base, while sealing edge of the piezoceramic transducer. Meanwhile, the flexible sleeve sealing the module and the nebulizer body. The module is simple and low cost.

### TECHNICAL FIELD

This utility model patent involves the technology of medical equipment, specifically, a nebulizer and its nebulization module.

### BACKGROUND ART

Nebulizer is an important medical device used for bronchial disinfection and treatment. Its main function is to generate mist-like particles from the medication and deliver them through a catheter to the affected area of the human body. Due to the direct reach of the medication to the affected area, the medication can be absorbed easily. The nebulizer mainly relies on a piezoceramic transducer to atomize the medication. However, as the apertures on the piezoceramic transducer is only between 0.5 μm and 5 μm, it is very prone to blockage problems, making the nebulizer inoperable. The piezoceramic transducer or the whole nebulization module have to be replaced in maintenance.

For example, a handheld nebulizer with application Ser. No. 20119214690369 and a handheld medical nebulizer with application Ser. No. 2019206754509 have to be completely disassembled in maintenance for replacement of the entire nebulization module. The base, piezoceramic transducer and the seals of the nebulization components have to be reinstalled one by one to form a new nebulization module. The maintenance process of the nebulization module is quite complicated, making the operation cost of the nebulizer higher.

### THE DETAILS OF THE UTILITY MODEL PATENT

For solving the problems existing in the current nebulization technology, this utility model patent provides with a nebulizer and its nebulization module. The piezoceramic transducer is mounted in the base by a flexible sleeve, greatly simplifying the structure of the nebulization module and facilitating the dismounting, assembling and maintenance of the piezoceramic transducer.

This utility model is embodied by the following technical solutions:

A kind of nebulization module including a base, a flexible sleeve and a piezoceramic transducer.

The flexible sleeve is attached on the edge of the piezoceramic transducer. The base has a mounting chamber where the flexible sleeve is installed. An electrode holder is provided on the outer wall of the base. The conductive pins in the holder are connected to the piezoceramic transducer according to the polarity.
Preferred,—a clamping structure is set in the base for fixing the flexible sleeve.
Preferred,—the clamping structure consists of a retaining ring, which is set on the inner wall of the base. The retaining ring is snapped on the flexible sleeve.
Preferred,—a coaxial snap ring is set on one side of the flexible sleeve. The outer wall of the snap ring is provided with an annular groove that matches with the retaining ring.
Preferred,—the inner wall of the flexible sleeve is provided with an annular groove, and the edge of the piezoceramic transducer is mounted in the annular groove.
Preferred,—a electric wire slot is provided on the flexible sleeve. The electric wires are laid in the slot. The both ends of the electric wires are connected with the piezoceramic transducer and the conductive pins, respectively.
Preferred,—the coaxial conductive pin installation holes are provided on both sides of the electrode holder.
Preferred,—two conductive pin installation holes are spaced apart at the top of the electrode

holder. The axial directions of the conductive pin installation holes are parallel to that of the piezoceramic transducer.
Preferred,—two conductive pin installation holes are spaced apart at both ends of the electrode holder. One end of both conductive pin installation holes extends toward the direction of the piezoceramic transducer.
   A kind of Nebulizer, Including the Nebulizer Body and the Nebulization Module Installed Inside the Nebulizer.

Compared with the existing technologies, this utility model has the following advantageous technical effects:

In the nebulization module, the edge of the piezoceramic transducer is enclosed by the flexible sleeve to form an integrated structure. The flexibility of the flexible sleeve allows itself to be mounted in the base, achieving a stable connection between the piezoceramic transducer and the base, while functioning as a seal for the edge of the piezoceramic transducer. When assembling the nebulization components, just install the whole module on the nebulizer body. At the same time, the flexible sleeve functions as a seal of the nebulization module and the nebulizer body. The nebulization module is simple in structure, which reduces the manufacturing cost, improves the assemble and disassemble efficiency and lowers down the maintenance cost of the nebulizer.

### DESCRIPTION OF THE ATTACHED DRAWINGS

FIG. 1 is the schematic diagram of the structure of the nebulization module in embodiment 1 of this utility model.

FIG. 2 is the schematic diagram of the structure of the flexible sleeve in this utility model.

FIG. 3 is the schematic diagram of the structure of the bottom of the flexible sleeve in this utility model.

FIG. 4 is the schematic diagram of the structure of the base in this utility model.

FIG. 5 is the schematic diagram of the structure of the piezoceramic transducer in this utility model.

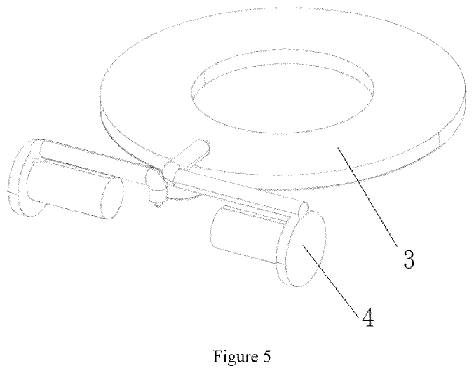

FIG. 6 is the schematic diagram of the structure of the nebulization module in embodiment 2 of this utility model.

FIG. 7 is the schematic diagram of the bottom structure of the nebulization module in embodiment 2 of this utility model.

FIG. 8 is the schematic diagram of the structure of the nebulization module in embodiment 3 of this utility model.

In these Figures: 1. base; 2. flexible sleeve; 3. piezoceramic transducer; 4. conductive pin; 5. electrode holder; 6. snap ring; 7. annular groove; 8. electric wire slot; 9. electrode hole; 10. retaining ring; 11. electric wire hole.

### EMBODIMENTS OF THE UTILITY MODEL

The detailed descriptions of this utility model against the attached drawings are given below, which is just an explanation rather than a limitation of this utility model.

A kind of nebulization module, including base (1), flexible sleeve (2) and piezoceramic transducer (3).

The flexible sleeve (2) is attached on the edge of the piezoceramic transducer (3). The base (1) has a mounting chamber where the flexible sleeve is installed. On the outer wall of the base (1) there is an electrode holder (5). The conductive pins (4) in the holder (5) are connected to the piezoceramic transducer according to the polarity.

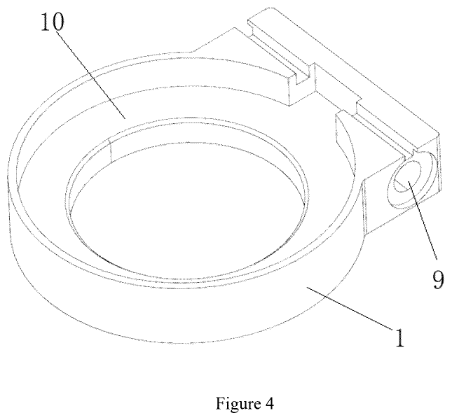

As shown in FIG. 4, the base is a circular structure; a retaining ring (10) is set on the inner wall of the base, and the flexible sleeve is installed in the mounting chamber of the base and snapped on the retaining ring; an electrode holder is provided on the outer wall of the base; there are two conductive pin installation holes in the electrode holder (5) and an electric wire slot on the surface of the holder; two conductive pins are installed in the conductive pin installation holes; both conductive pins are connected to the piezoceramic transducer (3) with electric wires; the electric wires are laid in the wire slot.

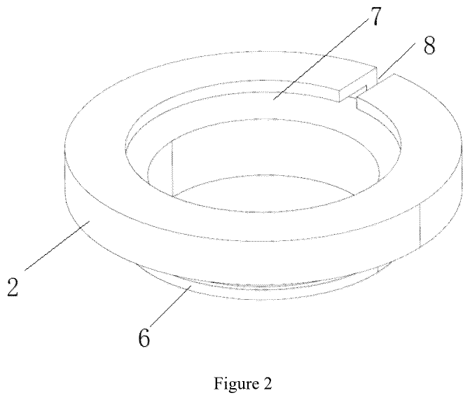

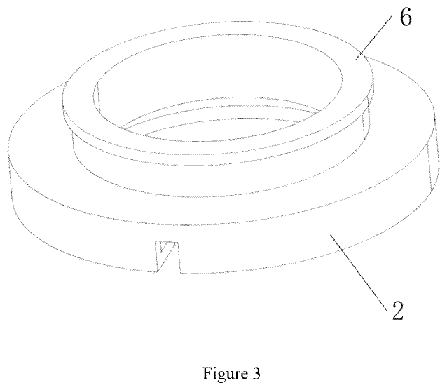

As shown in FIGS. 2 and 3, the flexible sleeve is made of silicagel, including a circular sleeve and a snap ring (6); the snap ring is coaxially installed at the bottom of the circular sleeve; there is an annular groove (7) on the inner wall of the circular sleeve, where the edge of the piezoceramic transducer is embedded. The piezoceramic transducer operates in the inner hole of the circular sleeve. This arrangement not only achieves the fixation of the piezoceramic transducer, but also allows the liquid to pass through the piezoceramic transducer without interfering its nebulization function. The snap ring is made of hollow tube, which is installed at the bottom of the circular sleeve and extends coaxially. On the side wall of the hollow tube there is an annular groove, which matches with the retaining ring. The circular sleeve is installed in the base, located at the top of the retaining ring. The hollow tube is deformed by squeeze to pass through the inner hole of the retaining ring, and then restored and locks the retainer ring. This way, the flexible sleeve is firmly fixed on the base.

On the circular sleeve there is an electric wire slot, which is interconnected with the annular groove (7). The electric wires of the piezoceramic transducer go through the wiring slot to the electrode holder and are connected on the conductive pins.

The flexible sleeve is made of medical silicagel, which ensures the sufficient elasticity of the sleeve and meets the operational requirements of the nebulizer.

As different types of nebulizer have different contact locations of the conductive pins, in order to make the nebulization module to adapt to different nebulizers, the locations of the conductive pins must meet the requirements of various kinds of nebulizers. Taking the following examples to make explanation on the locations of the conductive pins:

### Embodiment 1

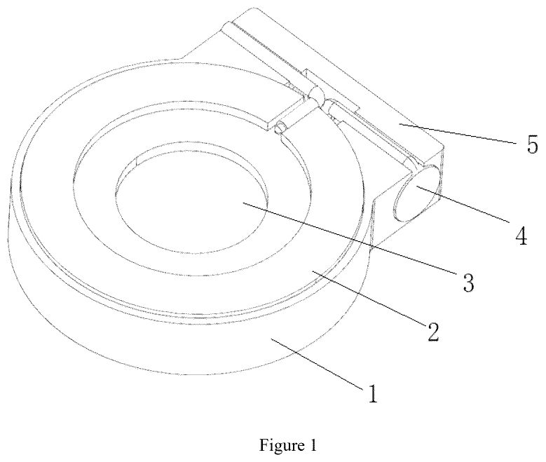

As shown in FIGS. 1 and 4, it is an nebulization module, including a base, a flexible sleeve installed inside the base and a piezoceramic transducer embedded inside the flexible sleeve. On the outer wall of the base an electrode holder (5) is provided. On both ends of the electrode holder (5) there are two conductive pin installation holes arranged coaxially. Two conductive pins are fixed in the installation holes. The both ends of the electric pins extend beyond the holder to connect with the contacts of the nebulizer for electrical connection. On the center line of the top of the electrode holder there is a groove. One end of each wire is connected to the conductive pin, respectively. The other ends of the two wires are laid in the groove and connect to the steel sheet and the ceramic sheet of the piezoceramic transducer, respectively. The steel sheet has a conductive pin, which extends into the groove via a wiring slot to connect with the electric wire. This arrangement can avoid any possible clearance between the steel sheet and the annular groove caused by soldering spots, thus the sufficient contact between the steel sheet and the bottom of the annular groove can be ensured for improvement of the seal performance.

### Embodiment 2

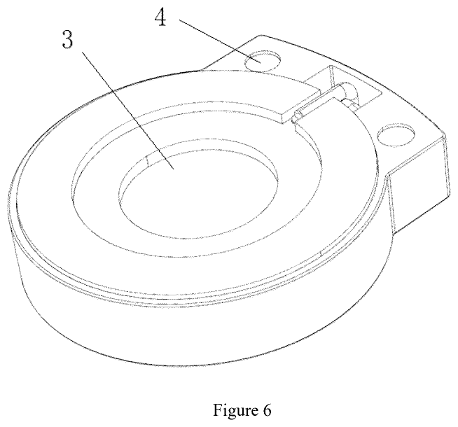

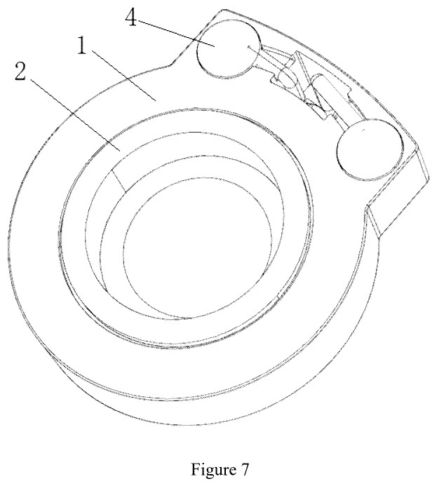

As shown in FIGS. 6 and 7, it is an nebulization module, including a base, a flexible sleeve installed inside the base and a piezoceramic transducer embedded inside the flexible sleeve. On the outer wall of the base there is an electrode holder (5). On the center line of the top of the electrode holder (5) there are two conductive pin installation holes arranged symetrically. The axial line of the installation holes is parallel to that of the base. The wiring slot is arranged at the bottom of the electrode holder (5). The two electric wires are connected with the piezoceramic transducer after going through the wiring slot.

### Embodiment 3

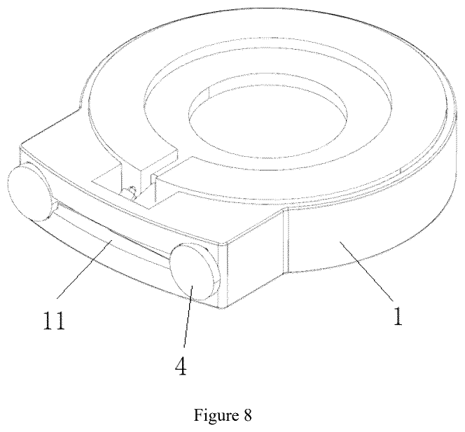

As shown in FIG. 8, it is an nebulization module, including a base, a flexible sleeve installed inside the base and a piezoceramic transducer embedded inside the flexible sleeve. On the outer wall of the base there is an electrode holder (5). On the center line of side surface of the electrode holder (5) facing the base there are two conductive pin installation holes arranged symetrically. The axial lines of the installation holes are arranged toward the plane surface of the piezoceramic transducer. The ends of the conductive pins extend toward the piezoceramic transducer. On the side surface of the electrode holder (5) there is a wiring hole. The electric wires connecting with the conductive pins are laid in the wiring slot after going through the wiring hole and connected to the piezoceramic transducer.

### Embodiment 4

It is a kind of nebulizer, including the nebulizer body and the nebulization module as mentioned above. This nebulizer atomizes the medicinal solution using the mechanism of ultrasonic vibration. When the liquid medicine vibrates, small droplets are formed, and then under the action of ultrasound, the droplets are further refined into tiny particles.

All the contents specified above is only for the purpose of illustrating the technical concepts of this utility model patent. It can not be used to limit the scope of protection of this utility model. Any modifications made on the basis of the technical concepts and technical solutions specified in this utility model patent shall all fall within the scope of protection specified in the claims of this utility model patent.

1. A kind of nebulization module including a base, a flexible sleeve and a piezoceramic transducer. The flexible sleeve is attached on the edge of the piezoceramic transducer. The base has a mounting chamber where the flexible sleeve is installed. On the outer wall of the base there is an electrode holder. The conductive pins in the holder are connected to the piezoceramic transducer according to the polarity.

2. A kind of nebulization module as specified in claim 1, which is characterized by a clamping structure used to fix the flexible sleeve on the base.

3. A kind of nebulization module as specified in claim 2. The clamping structure is characterized by a retaining ring, which is set on the inner wall of the base. The retaining ring is snapped on the flexible sleeve.

4. A kind of nebulization module as specified in claim 3, which is characterized by a coaxial snap ring set on one side of the flexible sleeve. The outer wall of the snap ring is provided with an annular groove that matches with the retaining ring.

5. A kind of nebulization module as specified in claim 1, which is characterized by a fact that the inner wall of the flexible sleeve is provided with an annular groove, where the edge of the piezoceramic transducer is embedded.

6. A kind of nebulization module as specified in claim 1, which is characterized by a electric wire slot on the flexible sleeve. The electric wires are laid in the slot. The both ends of the electric wires are connected with the piezoceramic transducer and the conductive pins, respectively.

7. A kind of nebulization module as specified in claim 1, which is characterized by a fact that the coaxial conductive pin installation holes are provided on both sides of the electrode holder.

8. A kind of nebulization module as specified in claim 1, which is characterized by a fact that two conductive pin installation holes are spaced apart at the top of the electrode holder. The axial directions of the conductive pin installation holes are parallel to that of the piezoceramic transducer.

9. A kind of nebulization module as specified in claim 1, which is characterized by a fact that two conductive pin installation holes are spaced apart at both ends of the electrode holder. One end of both conductive pin installation holes extends toward the direction of the piezoceramic transducer.

10. A kind of nebulizer, which is characterized by the nebulizer itself and the nebulization module installed inside the nebulizer as specified in claim 1.
